SEQUENCE LISTING (19 sequences)

SEQ ID NO: 1 - DNA, 42 bp, Artificial
  synthetic
actggttagtggattgcgtagtacaacgccaccgatgcggtc

SEQ ID NO: 2 - DNA, 22 bp, Artificial
  synthetic
tcgatacggcaccatgatgcac

SEQ ID NO: 3 - DNA, 20 bp, Artificial
  synthetic
aggctgctgtggtcgtgcga

SEQ ID NO: 4 - DNA, 70 bp, Artificial
  synthetic
tcgcacgacctggcgtctcgtggtgtcttttgacaccacgagtttgtactacgcaatcctgccgtatcga

SEQ ID NO: 5 - DNA, 42 bp, Artificial
  synthetic
tcgcacgacctggcgttgtactacgcaatcctgccgtatcga

SEQ ID NO: 6 - DNA, 42 bp, Artificial
  synthetic
catacgcagtggatagcgaccaaccgttacaccgatgcggta

SEQ ID NO: 7 - DNA, 22 bp, Artificial
  synthetic
tagtgtcatcaccagttgtatc

SEQ ID NO: 8 - DNA, 20 bp, Artificial
  synthetic
gagcaatcgtggctgccgag

SEQ ID NO: 9 - DNA, 70 bp, Artificial
  synthetic
ctcggcagcctgtaacgctggcaacatttttatgttgccagcttggttggtcgctatcctgatgacacta

SEQ ID NO: 10 - DNA, 42 bp, Artificial
  synthetic
ctcggcagcctgtaacggttggtcgctatcctgatgacacta

SEQ ID NO: 11 - DNA, 99 bp, Artificial
  synthetic
cattgatatctactggatacaactggactgcgtatgtaccgcatcggacgattgctcgtgcatcatggactaaccagtgaccgcatcggacagcagcct

SEQ ID NO: 12 - DNA, 36 bp, Artificial
  synthetic
cagtagatatcaatgctaatgtttbtttbtcattag

SEQ ID NO: 13 - DNA, 42 bp, Artificial
  synthetic
gtagtatcgtggctgtgtaatcagcgcggcaccaactggcat

SEQ ID NO: 14 - DNA, 22 bp, Artificial
  synthetic
gtcaatgctcaccgattcaacc

SEQ ID NO: 15 - DNA, 20 bp, Artificial
  synthetic
cgccgttagtggatgtcgcc

SEQ ID NO: 16 - DNA, 42 bp, Artificial
  synthetic
ggcgacatcctgccgcgctgattacacagcctgagcattgac

SEQ ID NO: 17 - DNA, 141 bp, Artificial
  synthetic
cattgatatctactggatacaactggactgcgtatgtaccgcatcggacgattgctcgtgcatcatggactaaccagtgaccgcatcggacagcagcctggttgaatcggacgatactacatgccagttggactaacggcg

SEQ ID NO: 18 - DNA, 141 bp, Artificial
  synthetic
cattgatatctactggatacaactggactgcgtatgtaccgcatcggacgattgctcggttgaatcggacgatactacatgccagttggactaacggcggtgcatcatggactaaccagtgaccgcatcggacagcagcct

SEQ ID NO: 19 - DNA, 141 bp, Artificial
  synthetic
cattgatatctactgggttgaatcggacgatactacatgccagttggactaacggcggatacaactggactgcgtatgtaccgcatcggacgattgctcgtgcatcatggactaaccagtgaccgcatcggacagcagcct

(19) United States
(12) Patent Application Publication
(10) Pub. No.: US 2008/0221315 A1
(43) Pub. Date: Sep. 11, 2008

(54) NUCLEIC ACID-BASED TRANSLATION SYSTEM AND METHOD FOR DECODING NUCLEIC ACID ENCRYPTED MESSAGE

(75) Inventors: Alejandra V. Garibotti, Barcelona (ES); Shiping Liao, New York, NY (US); Nadrian C. Seeman, New York, NY (US)

Correspondence Address: BROWDY AND NEIMARK, P.L.L.C.;624 NINTH STREET, NW, SUITE 300, WASHINGTON, DC, 20001-5303, US

(73) Assignee: New York University, New York, NY (US)

(21) Appl. No.: 11/950,723

(22) Filed: Dec. 5, 2007

Related U.S. Application Data
(60) Provisional application No. 60/869,045, filed on Dec. 7, 2006.

(51) Int. Cl. C07H 21/02

(52) U.S. Cl. 536/23.1

(57) ABSTRACT

A nucleic acid-based translation system where the components of a nucleic acid multicrossover molecule serve as message, translation device and part of the translated product. One continuous strand of a nucleic acid multicrossover molecule acts as a message, which nucleic acid crossover strands, functioning together as a translation device, translate into nucleic acid product strands. Organic molecules appended to the backbone of the nucleic acid product strands can also be polymerized to form a polymer sequence of appended organic molecules.

### CROSS-REFERENCE TO RELATED APPLICATIONS

This application claims priority from provisional application 60/869,045, filed Dec. 7, 2006, the entire contents which are hereby incorporated by reference.

### GOVERNMENT LICENSE RIGHTS

The experiments reported in this application were supported in part by: the National Institute of General Medical Sciences, grant no. GM-29554; the National Science Foundation, grant nos. DMI-0210844, EIA-0086015, CCF-0432009, CCF-0523290, CTS-0548774 and CTS-0608889; and Army Research Office, grant no. 48681-EL. The U.S. Government has a paid-up license in this invention and the right in limited circumstances to require the patent owner to license others on reasonable terms as provided for by the terms of the above grants.

### BACKGROUND OF THE INVENTION

1. Field of the Invention

The present invention relates to a system and method for translating DNA signals into polymer assembly instructions and to cryptography.

2. Description of the Related Art

Recently, the laboratory of the present inventors reported combining two sequence-dependent robust DNA-based 2-state nanomechanical devices with DNA parallelogram motifs to produce a translation machine (Liao et al., 2004; US2006-0035255). A picture of the device is shown in FIG. 1A. The important parts of the machine are the gaps at the top, flanked by numbers that represent sticky ends. A series of DAE-type DNA double crossover molecules (Fu et al., 1993) are used in this system to emulate aminoacyl-tRNA molecules. Their top strand corresponds to the amino acid, and the bottom domain of the DX molecules contains sticky ends complementary to the sticky ends in the gaps. The independently-addressable 2-state devices switch the components flanking the gaps, so that four different translation products can be produced, depending on the states of the two devices. One weakness of this device is that it is a complex DNA construct for the current state of the art; another weakness is that it is a rotationally-based linear system, so that the size of the machine must be similar to the size of the product. In a different vein, another group has reported a system that entails more complex chemistry, but is conceptually much simpler (Endo et al., 2005). They have used two different strands of DNA coupled via their phosphates in an unusual linkage as the basis of a translation system; these strands link a ‘message’ strand and a ‘product’ strand. The system does not enforce the directionality of the product, because of possible swiveling around the unusual linkage.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a nucleic acid-based translation system where the components of a nucleic acid multicrossover molecule serve as message, translation device and part of the translated product. One continuous strand of a nucleic acid multicrossover molecule acts as a message, which nucleic acid crossover strands, functioning together as a translation device, translate into nucleic acid product strands. Thus, a nucleic acid message in the form of a nucleotide sequence can be translated into an unrelated sequence. The unrelatedness of the nucleotide sequence of the nucleic acid product strands to the nucleotide sequence of the message strand can be carried further to the sequence of pendant organic molecules that are appended to the backbone of the nucleic acid product strands. The pendant organic molecules can be polymerized to form a polymer sequence of such appended organic molecules.

The present invention also provides a method for synthesizing a polymer sequence of organic molecules using the nucleic acid-based translation system of the present invention.

Further provided by the present invention is a method for decoding an encrypted message on a nucleic acid strand using the components of a nucleic acid multicrossover molecule as the encrypted message, decoder keys and the decoded message.

### BRIEF DESCRIPTION OF THE DRAWINGS

FIGS. 1A-1E show schematic drawings of DNA DX molecules. FIG. 1A is a prior art nanomechanical translation apparatus. The Arabic numbers refer to sticky ends, and the Roman numerals indicate two independently addressable DNA-based nanomechanical devices. Double crossover (DX) molecules (analogous to aminoacyl tRNA molecules in protein synthesis) bind to the upper sticky ends, depending on the states of the devices. The setting of the devices shown would bind DX molecules flanked by sticky ends complementary to 1 and 2 and to 4 and 6. Switching the state of Device II (flipping 6 and 7), for example, would bind DX molecules complementary to 1 and 2 and to 4 and 7. Four states are available. FIGS. 1B and 1C show two types of antiparallel double crossover molecules, DAE (FIG. 1B), with an even number of double helical half-turns between the crossover, and DAO (FIG. 1C), with an odd number of half-turns between the crossovers. The DAE molecule as illustrated contains five strands, two of which are continuous, or helical strands, and three of which are crossover strands, including the cyclic strand in the middle. The 3′ ends of each strand are indicated by an arrowhead. The DAO molecule is depicted in FIG. 1C, and it contains only 4 strands. The twofold symmetry element is perpendicular to the page, vertically, for the DAE molecules, and it is horizontal within the page, for the DAO molecule. FIG. 1D is another view of a DAE-type DX molecule. 3′ ends are indicated by arrowheads; the elliptical symbol at the center indicates the backbone dyad symmetry. The two continuous strands can function as a coded message and as the decoded translation product. The three crossover strands act as the translation apparatus, which decodes the message. Fig. BE is a schematic of this DNA translation apparatus which translates the coded DNA message found in one continuous strand to a decoded DNA translation product.

FIGS. 2A-2C show schematic drawings of three types of parallel double crossover molecules, DPE (even number of double helicial half-turns between crossovers; FIG. 2A), DPON (odd number of double helical half turns between crossovers with a turn and a half containing one major groove spacing and two minor groove spacings; FIG. 2B) and DPOW (odd number of double helical half turns between crossovers with a turn and a half containing one minor groove spacing and two major groove spacings; FIG. 2C).

FIG. 3 shows a schematic drawing of a triple crossover (TX) molecule.

FIGS. 4A-4E show schematic drawings of the systems used the Example hereinbelow. FIG. 4A shows an unsuccessful system containing hairpins in the product structures. The hairpins interfered with PCR, and were abandoned. FIG. 4B shows a simpler and successful two-component system. There is a single 84-mer strand (DAB09) at the bottom acting as the message, and two product 42-mer strands (DA04S and DB08S) shown before ligation. The translation strands with the crossovers in them are shown as well. The double crossover (DX) nature of the molecules (fused by strand DAB09) is evident from the drawing. The space between the two helical domains of the DAE molecules is exaggerated for clarity; there is only a single nucleotide backbone linkage between them. The biotin groups in the hairpin on the right are not used. FIGS. 4C-4E show three-component systems analogous to the two-component system shown in FIG. 4B. The sequences of the strands used in FIGS. 4A-4E are identified below as follows: DA01 (SEQ ID NO:1), DA02 (SEQ ID NO:2), DA3 (SEQ ID NO:3), DA4 (SEQ ID NO:4), DA04S (SEQ ID NO:5), DB05 (SEQ ID NO:6), DB06 (SEQ ID NO:7), DB07 (SEQ ID NO:8), DB08 (SEQ ID NO:9), DB08S (SEQ ID NO:10), DAB09 (SEQ ID NO:11), DBiotin (SEQ ID NO:12), DC01 (SEQ ID NO:13), DC02 (SEQ ID NO:14), DC03 (SEQ ID NO:15), DC05S (SEQ ID NO:16), CAB10 (SEQ ID NO:17), ACB11 (SEQ ID NO:18), and ABC12 (SEQ ID NO:19).

FIGS. 5A and 5B are denaturing gels showing the products of ligation. FIG. 5A shows the ligation products corresponding to the molecule shown in FIG. 4B. A 50-mer linear marker lane (L50) is shown at the right. The target 84-mer is the major ligation product visible in the lane (AB) containing ligation products. FIG. 5B shows the ligation products corresponding to the molecules in FIGS. 4C-4E. A 10-mer linear marker lane (L10) is shown at the left. The products of systems CAB (FIG. 4C), ACB (FIG. 4D) and ABC (FIG. 4E) are shown at the right. Dimer 84-mer molecules are visible. The ratio of 84-mers to target 126-mer products (126-P) are roughly 55:45 (CAB) and 63:37 (ACB and ABC). The message strand (141-M) is indicated as well.

FIGS. 6A-6B show non-denaturing gels of the triple combinations of messages and pre-ligation products. Both FIGS. 6A and 6B contain linear markers separated by 10 nucleotide pairs. The products have a mobility in the vicinity of their total mass, which is 278 nucleotide pairs. The single band seen is a clear indicator of the stability of the triple-DX complex.

FIG. 7 schematically shows polymerization of a sequence of organic molecules appended from the backbone of a nucleic acid antiparallel double crossover molecule (DAE). The different squares represent different moieties in the polymer sequence of the appended organic molecules. The open head and tail of the arrows at the top of the figure represent compatible reactive groups which react to form a covalent bond.

FIG. 8 schematically shows polymerization of a sequence of organic molecules appended from the backbone of a nucleic acid parallel double crossover molecule with an even number of double helical half turns between crossovers (DPE). The different squares represent different moieties in the polymer sequence of the appended organic molecules. The open head and tail of the arrows represent compatible reactive groups which react to form a covalent bond.

FIG. 9 schematically shows polymerization of a sequence of organic molecules appended from the backbone of a nucleic acid parallel double crossover molecule with an odd number of double helical half turns between crossovers and with a turn and a half containing one major groove spacing and two minor groove spacings (DPON). The different squares represent different moieties in the polymer sequence of the appended organic molecules. The open head and tail of the arrows represent compatible reactive groups which react to form a covalent bond.

FIG. 10 schematically shows polymerization of a sequence of organic molecules appended from the backbone of a nucleic acid parallel double crossover molecule with an odd number of double helical half turns between crossovers and with a turn and a half containing one minor groove spacing and two major groove spacings (DPOW). The different squares represent different moieties in the polymer sequence of the appended organic molecules. The open head and tail of the arrows represent compatible reactive groups which react to form a covalent bond.

FIG. 11 schematically shows polymerization of a sequence of organic molecules appended from the backbone of a nucleic acid triple crossover molecule (TX). The different squares represent different moieties in the polymer sequence of the appended organic molecules. The open head and tail of the arrows represent compatible reactive groups which react to form a covalent bond.

### DETAILED DESCRIPTION OF THE INVENTION

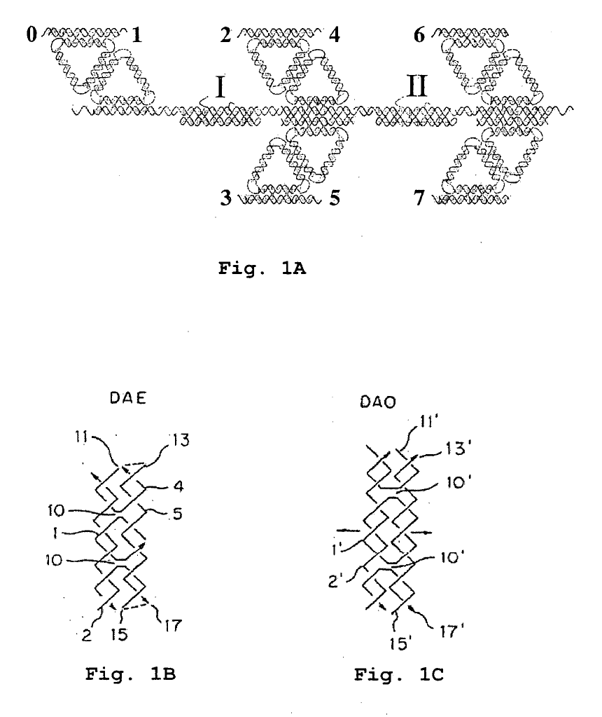

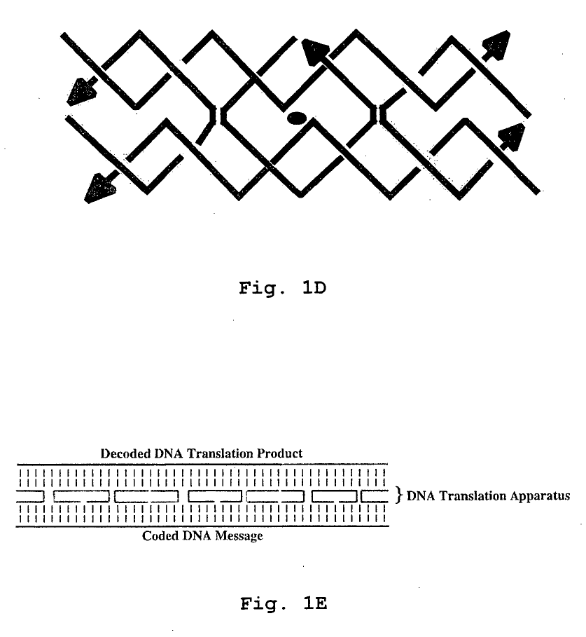

The present inventors have developed a translation system using nucleic acid double crossover (DX) molecules that generates unique products (FIG. 1E). This particular species of DX molecule is called a DAE molecule (Double Crossover Anti-parallel Even) and contains an even number of half-turns between crossover points, so there is a continuous strand on both sides of the molecule. One of these strands acts as the input strand containing the message, and a second strand acts as the output (product of translation). The crossover strands carry the ‘code’ that connects the two sides of the molecule. This system is both more robust and simpler than previous DNA-based translation systems that have been reported. It is designed to be useful in a variety of applications that utilize the concept of translating from one code to another.

DNA molecules containing two crossover sites between helical domains have been widely suggested as intermediates in recombination processes involving double stranded breaks.

Accordingly, “double crossover molecules” are those nucleic acid molecules containing two branched junctions (Holliday junctions corresponding to the crossover sites) linked together by ligating two of their double helical arms. By branched junction is meant a point from which three or more helices (arms) radiate.

There are five isomers of double crossover molecules (Fu et al., 1993), which fall into two broad classes of molecules differentiated by the relative orientations, parallel (DP) or antiparallel (DA), of their helical axes.

In order to avoid torsional stress on the double crossover molecules, the distance between the branched junction or crossover points are specified as either even (E) or odd (O) multiples of half helical turns. Antiparallel double crossover molecules with an even number of half helical turns between crossover points are designated DAE and those with an odd number are designated DAO. FIGS. 1B and 1C show schematic representations of the DAE and DAO forms, respectively, of antiparallel double crossover molecules in which two strands of a helix are presented as a pair of lines. The DAE and DAO molecules depicted in FIGS. 1B and 1C have strands 1, 2, 4 and 5 and strands 1′ and 2′, respectively. There are two half helical turns between the two crossover points (10) in the DAE molecule depicted and three half helical turns between crossover points (10′) in the DAO molecule.

The simplicity of the Endo et al. system is now combined with the simplicity of DNA synthesis to produce a translation system that is unidirectional and easy to access. It is not necessary to do anything more complex than ordering oligonucleotides from a DNA synthesis facility or from a commercial vendor for the purpose of using this system for decoding encrypted nucleic acid messages. A most preferred embodiment of this system is the DAE isomer of the DNA double crossover (DX) molecule. DNA double crossover molecules contain two DNA double helical domains that are linked to each other by two Holliday-like (Holliday, 1964) crossover points (Fu et al., 1993). In addition to their use in the rotary translation system described above in the Description of the Related Arts section, DX molecules have been used in nanoconstruction of geometrical objects and lattices (U.S. Pat. No. 6,072,044) to build 2D periodic arrays (Winfree et al., 1998; U.S. Pat. No. 6,255,469), as components of nanomechanical devices (Mao et al., 1999), in algorithmic assemblies (Rothermund et al., 2004), and in assembling DNA nanotubes (Rothermund et al., 2004). There are a variety of DX molecules, but those in which the crossovers occur between strands of opposite polarity (antiparallel) are the most robust when the separations between the crossovers are short, say two turns of DNA or less (Fu et al., 1993). These crossover points can be separated by an even (DAE) or odd (DAO) number of half-turns of the DNA double helix. Those separated by an even number of turns (DAE) lead to molecules that contain continuous strands in both helical domains. Most preferably, the double crossover molecules in the translation system of the present invention are DAE double crossover molecules.

The schematic diagram in FIG. 1D shows a DAE molecule whose crossovers are separated by a single turn of DNA. There are two continuous strands, one on each side of the DAE molecule. One can serve as the message and the other as the translation product (FIG. 1E). The translation apparatus then is composed of the three strands (crossover strands) that connect the two continuous strands; in other words, strands that recognize both of the two continuous strands act as the translation units (see also FIG. 1E). Without the crossover strands to act as translation units, it would be impossible to decode the information from one strand into another strand. Once the crossover strands are known, translation is trivial. If one designates, say, the bottom continuous strand as the message strand and the top continuous strand as the product strand, one can establish a code or correspondence relating the two by selecting the sequences of the crossover strands that connect them. The parts of the crossover strands that bind to the lower strand select a particular message strand, and the parts that bind to the upper strand select a particular product strand for which it codes. There is no need to limit the message strand to a strand from a single DAE unit; it could be a single long continuous strand from a multimer of DAE units (or more generally, from a multimer of multicrossover units). Indeed, the naturally-occurring messages that are used in protein synthesis (mRNA molecules) are long continuous strands that code for the entire length of a protein polymer. In the work reported in the Example hereinbelow, the present inventors have used message strands that are two or three DAE units long. Thus, the present inventors are directing a specific product (sequence or chain of product strands) from a particular message encrypted for all of those component product strands.

It is important to realize that this system does not require a continuous message, but could be expected to work with disjoint message segments that spanned two different nucleic acid multicrossover units; the advantage of disjoint messages is that the entire message need not be determined at once, and the product can be used to describe the history of an evolving or oscillating system. In addition to making new DNA sequences, pendent polymers (i.e., Zhu et al., 2003) can be introduced in the product strands to produce interesting polymers. Thus, this system allows the simple recasting of a message into another form of chemistry. For example, if the product strands contain pendant polymers, the message strand can encrypt instructions to produce a particular polymer sequence.

One aspect of the present invention is directed to a nucleic acid-based translation system which includes as its components, a nucleic acid template that serves as a message, a plurality of nucleic acid product strands with or without organic molecules (pendant molecules) appended to the backbone of the nucleic acid product strands, and a plurality of nucleic acid crossover strands capable of forming, with the nucleic acid template strand and the nucleic acid product strands, at least one nucleic acid multicrossover molecule. Thus, the components of the nucleic acid-based translation system are essentially the components of nucleic acid multicrossover molecules, where the nucleic acid crossover strands function as translation units of a translation device to translate the “message”, which is the nucleotide sequence of the nucleic acid template strand, into a sequence of nucleic acid product strands. These nucleic acid product strands optionally have pendant molecules (i.e., organic molecules appended to the backbone of the product strands and serving as monomeric units) with compatible reactive groups. This sequence of nucleic acid product strands can be ligated together into a chain of nucleic acid product strands. When organic molecules with compatible reactive groups are appended to the backbone of the nucleotide acid product strands, they can be reacted/polymerized by their compatible reactive groups to form a polymer sequence of organic molecules (FIGS. 7-11). This polymer can remain appended to the nucleic acid product strands or it can be cleaved off and separated from the translation system as a translation product. It is preferred that the pendant (appended) organic molecules are a mixture of different organic molecules with compatible reactive groups which can be polymerized into a polymer sequence of monomeric units.

It should be appreciated that the nucleic acid template strand in the nucleic acid-based translation system of the present invention can serve as the template strand of not only one nucleic acid multicrossover molecule but as a template strand for a plurality of nucleic acid multicrossover molecules sharing this strand. The nucleic acid-based translation system of the present invention also encompasses the situation where there are more than one template strand, such as when disjoint message segments (template strands) are used in the translation system as a processive message.

Each of the at least one nucleic acid multicrossover molecules formed in the present nucleic acid-based translation system can be described as having a first and second helices that are parallel to each other, where each of the first and second helices has a unidirectional nucleic acid strand disposed along its respective helical axis or alternatingly disposed along the helical axes of both of said first and second helices. One of the unidirectional nucleic acid strand acts as the nucleic acid template strand and the other unidirectional nucleic acid strand contains the plurality of nucleic acid product strands either ligated together or capable of being ligated together into a continuous unidirectional strand. Thus, the nucleic acid crossover strands that translate the nucleic acid template strand into a plurality of nucleic acid product strands anneal with both the nucleic acid template strand and the plurality of nucleic acid product strands to form at least one nucleic acid multicrossover molecule.

As used herein, a “unidirectional” nucleic acid strand is a nucleic acid strand, which when considered in the conventional 5′ to 3′ direction, follows a single direction parallel to a helical axis. A nucleic acid strand is considered “unidirectional” even if the strand crosses over to another parallel helix as long as it still follows the same direction and does not loop back in the opposite direction.

Also as used herein, a “nucleic acid crossover strand” is any strand in a nucleic acid multicrossover molecule which crosses over from one double helix to another and which is not either the nucleic acid template/message strand or one of the nucleic acid product strands.

Preferably, the nucleic acid multicrossover molecule is either a nucleic acid double crossover or triple crossover molecule (FIG. 3). When it is a nucleic acid triple crossover molecule, the nucleic acid-based translation system further includes a nucleic acid strand that link together the nucleic acid template strand, the plurality of nucleic acid product strands and the plurality of nucleic acid crossover strands into at least one nucleic acid triple crossover molecule. It should be appreciated from FIG. 3 that a triple crossover (TX) molecule has three parallel helices and continuous unidirectional strands in each of the three helices. Thus, any two of the continuous unidirectional strands can serve as the input and output of the nucleic acid-based translation system.

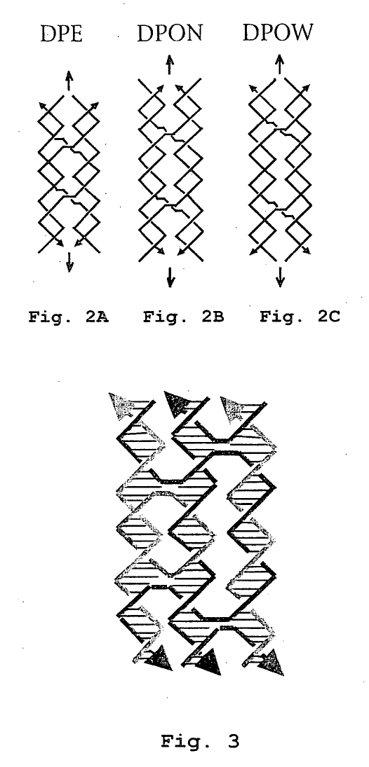

When the nucleic acid multicrossover molecule is a double crossover molecule, it is preferably a DAE (antiparallel double helices, even number of half helical turns between crossovers; FIGS. 1B and 1D), DPE (parallel double helices, even number of half helical turns between crossovers; FIG. 2A), DPON (parallel double helices, odd number of half helical turns between crossovers with a helical turn and a half containing one major groove spacing and two minor groove spacings; FIG. 2B), or DPOW (parallel double helices, odd number of half helical turns between crossover with a helical turn and a half containing one minor groove spacing and two major groove spacings; FIG. 2C), more preferably DAE or DPE and most preferably DAE.

Another aspect of the present invention is a method for synthesizing a polymer sequence of organic molecules which involves operating the nucleic acid-based translation system to produce a sequence of nucleic acid product strands with organic molecules appended to their backbones. The appended organic molecules are then polymerized together by their compatible reactive groups to synthesize an appended polymer sequence of organic molecules. The nucleic acid product strands can also be ligated together to form a continuous chain of nucleic acid product strands. Furthermore, the appended polymer sequence of organic molecules can also be cleaved from the nucleic acid product strands to release the polymer sequence.

A further aspect of the present invention is a method for decoding an encrypted message on a nucleic acid strand by using the nucleic acid-based translation system of the present invention. This method involves adding a set of nucleic acid crossover strands as decoder keys and a set of nucleic acid product strands as “decoded” unidirectional nucleic acid message strands to an encrypted nucleic acid message strand which contains the encrypted message in the form of the nucleotide sequence of the nucleic acid message strand. The decoder keys to decode the encrypted message are nucleic acid crossover strands that can anneal to both the encrypted nucleic acid message strand and the decoded nucleic acid message strands. By annealing the nucleic acid crossover strands as decoder keys to the encrypted nucleic acid message strand and the decoded nucleic acid message strands, at least one nucleic acid multicrossover molecule having two parallel helices (in one helix, the encrypted nucleic acid message strand; in the other helix, the one or more decoded nucleic acid message strands) are formed. The decoded message can then be determined from the decoded nucleic acid message strands. This method can alternatively involve ligating the decoded nucleic acid message strands into a continuous chain of decoded message strands before determining the decoded message. A further step may involve denaturing the at least one nucleic acid multicrossover molecule to release the continuous chain of decoded nucleic acid message strands before determining the decoded message.

Thus, the present inventors have shown that simple, commercially-available DNA sequences can be used to produce an encryption whose key is itself DNA. In an era when information on the internet can be used to produce a virus with unsophisticated DNA chemistry (Cello et al., 2002), it is shown here that a DNA message can be produced and translated (decoded) using the same basic approach. Without the key (the crossover strands of the nucleic acid multicrossover molecule, i.e., the central crossover strands of the DAE molecule), there is no simple way to break the code; with those strands, it is trivial to do so.

It should be appreciated that the terms “nucleic acid” or “polynucleic acid”, which can be used interchangeably, refer to both DNA and RNA and hybrids of the two, although preferably the “nucleic acid” is DNA. The structure need not resemble anything which can theoretically be made from nature.

A particular nucleic acid strand may employ bases other than the standard five, adenine, cytosine, guanine, thymine and uracil. Derivatized (e.g., methylated) and other unusual bases such as iso-guanine, iso-cytosine, amino-adenine, K, X, n, (Piccirilli et al., 1990), inosine and other derivatives of purine and pyrimidine may be used. A preferable feature in the selection of the bases is that they be capable of interacting with a base opposing them to form a specifically paired attraction. In natural DNA and RNA, hydrogen bonding forms this interaction. However, opposite ion charges, hydrophobic interactions and van der Waals forces may also be acceptable forms of interaction. These interactions expand the choices over naturally occurring bases to give a wider assortment of physical properties. Non-limiting examples of nucleic acids include DNA, RNA, Peptide Nucleic Acid (PNA), and Locked Nucleic Action (LNA). A review of some nucleic acid variations, including derivatized/modified bases and other unusual bases, is presented in Freier et al. (1997).

Within a particular strand, the heterocyclic base may be entirely missing from the sugar moiety. This may be particularly desirable where the strands bend, form a junction, or where one desires fewer forces holding the strands together.

A particular strand need not have a single contiguous ribose-phosphate or deoxyribose-phosphate backbone. One may employ a simple inorganic or organic moiety or polymeric spacer between segments of polynucleotide. Spacers such as polyethylene, polyvinyl polymers, polypropylene, polyethylene glycol, polystyrene, polypeptides (enzymes, antibodies, etc.) peptide nucleic acids (PNA), polysaccharides (starches, cellulose, etc.) silicones, silanes and copolymers, etc., may be employed. An example of such a hybrid structure is dodecadiol having phosphoramidite at one end. This structure has been inserted covalently instead of four T nucleotides to form a hairpin loop in a fashion similar to the nucleotides it replaces. See Mitchel J. Doktycz, Ph.D. Thesis (1991), University of Illinois, Chicago. The term “oligonucleotide”, “polynucleotide”, “polynucleic acid”, and “nucleic acid” are intended to cover all of these structures.

In nature and in the field of molecular biology, double stranded DNA generally occurs in the B form. However, for the purposes of this invention it may be desirable for DNA or other double stranded nucleic acids to exist in the A, C, D or Z form. Various bases, derivations and modifications may be used to stabilize the structure in the A, C, D or Z form as well.

From a chemical standpoint, the present inventors expect to be able to couple this system with a recent method that adds reactive groups to the backbone residues of nucleotides (Zhu et al., 2003; WO 05/001035 and U.S. patent application Ser. No. 10/855,893). As reported, that method adds bivalent reactive groups to each nucleotide in the backbone. Adding a reactive group, such as diamino groups or dicarboxyl groups, to the continuous chain to a few accessible sites (e.g., once per helical turn) would be independent of steric effects and can attach another detachable polymer. Such groups could be used in this context to scaffold the construction of diverse and unprecedented polymers of well-defined size and composition.

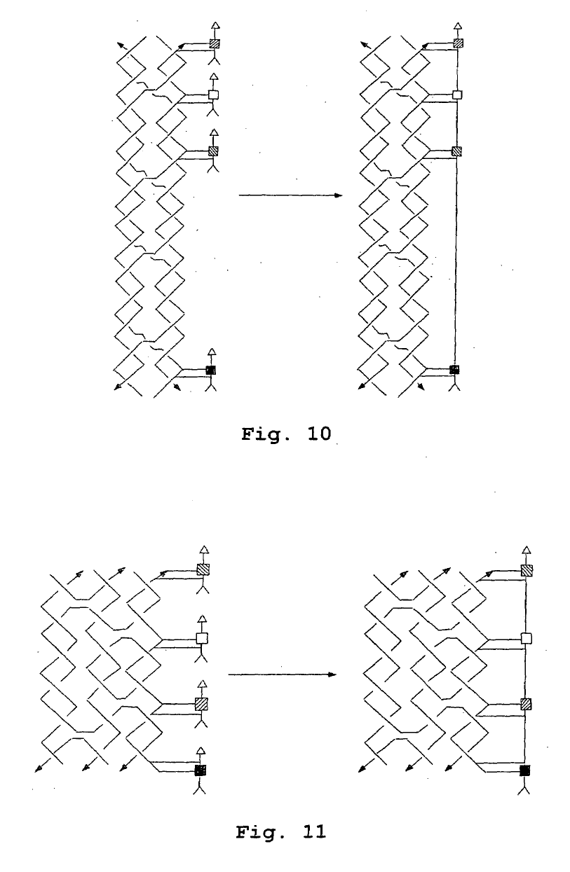

Construction of appended (pendant) organic polymers can be accomplished by assembly of smaller units on nucleic acid multicrossover molecules followed by polymerization templated by the nucleic acid molecules, as shown in FIG. 7 (DAE), FIG. 8 (DPE), FIG. 9 (DPON), FIG. 10 (DPOW) and FIG. 11 (TX). One strand (nucleic acid product strand) can be constructed with one covalent attachment per turn of, e.g., B-form DNA, for DAE, DPE and TX. Molecular models suggest that 34 Å/turn corresponds to approximately 30 atoms (e.g., 30 atoms in a fully anti-form alkane chain gives an end-to-end distance of 35 Å). Preferably, there are two covalent attachments (within a half-turn of the helix) to a nucleic acid product strand per monomeric unit of the appended (pendant) organic polymer molecule to prevent the monomeric unit from being able to assume more than one orientation (FIGS. 7-11). Cassettes containing 2′-deoxy-2′-alkylthiouridine can be incorporated into DNA strands by the methods developed in the Seeman and Canary laboratories (Zhu et al 2003; Zhu et al 2002). The nucleic acid multicrossover molecules can be assembled and the amides linked using peptide coupling chemistry. In the case of DPON (FIG. 9) and DPOW (FIG. 10), due to the nature of the product strand, which alternates between two helices, only the appended organic molecules along one helix are polymerized into one organic polymer. It should also be appreciated from the nature of DPON and DPOW molecules that two organic polymers can be synthesized, one appended on each helix where the product strand is alternatingly disposed.

The temptation by the DNA will determine the length of the organic polymer formed. Intermolecular reactions will be several orders of magnitude slower and will essentially not be observable under the conditions of the synthesis (Gartner and Liu, 2001). The DMT-MM reagent will activate all of the carboxyl groups including the terminal one, but the only available amines are either 260 Å away or in another molecule. In either case, no reaction except the background reaction with water to regenerate the carboxyl will occur. Coupling will occur only between adjacent amines and carboxylates, not between remotely located functional groups, due to the rigidity of the DAE molecule, which is even more rigid than duplex DNA (Sa-Ardyen et al., 2003).

Using these procedures, the first generation polymer 10 below can be produced where Q₁=Q₂=triethylene glycol and n is determined by the length of the nucleic acid message strand translated into nucleic acid product strands.

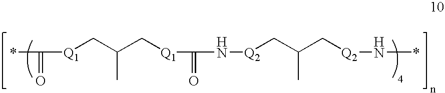

Various “monomeric units” can be prepared with varying Q moieties. Additionally, the monomer synthesis allows Q₁ and Q₂ to be different. Several examples of building blocks that may be used as Q moieties are shown below.

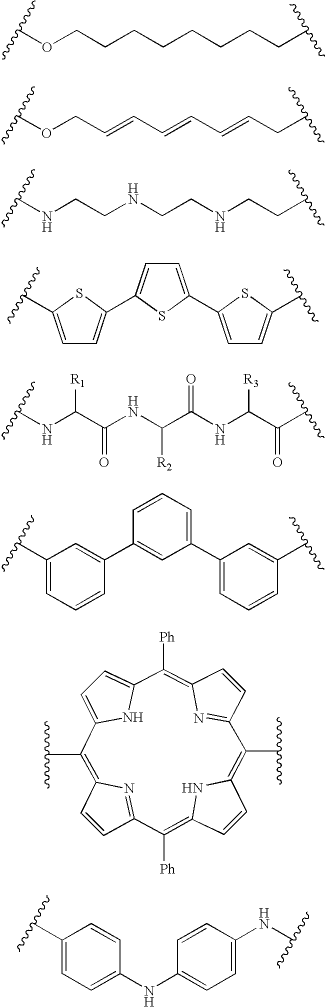

By constructing various monomeric units and using the nucleic acid-based translation system of the present invention, a polymer with generalized formula 12 can be obtained.

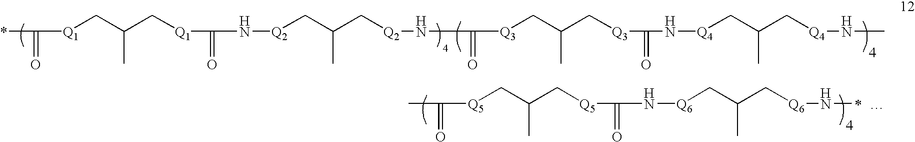

It is worth noting that the linkage chemistry is a point of potential variability. Additional chemistries are available for linking organic moieties together. A variety of organic reactions has been shown to be compatible with DNA (Kanan et al, 2004). In principle, such reactions could be used to link organic polymers, although they would need to be examined for compatibility in DNA automated synthesis.

In addition, the number of linkages to the nucleic acid multicrossover molecule can be varied. For example, the number of connections can be reduced to one every second turn by replacing the triethylene glycol with octaethylene glycol (Fluka) in the synthesis. The connections being at the same angular point (although not being limited to every 360° turn) of the multicrossover molecule. Peptide residues generated from automated synthesis are available in even greater lengths, making possible even fewer nucleic acid multicrossover molecule/polymer cross links. Even longer peptides are available using modern chemical ligation techniques (Bang and Kent, 2004). Artificial peptide residues can be incorporated into sequences generated by these protocols. The sulfide linker group could be derived from cysteine, such that after reductive cleavage of the peptide from the DNA, the cysteine residue would be converted into an alanine.

The ladder polymers (polymer of organic molecules appended to one or more nucleic acid product strands) capable of being assembled by the nucleic acid based translation system of the present invention are encompassed by the generic structure presented below as general formula (I).

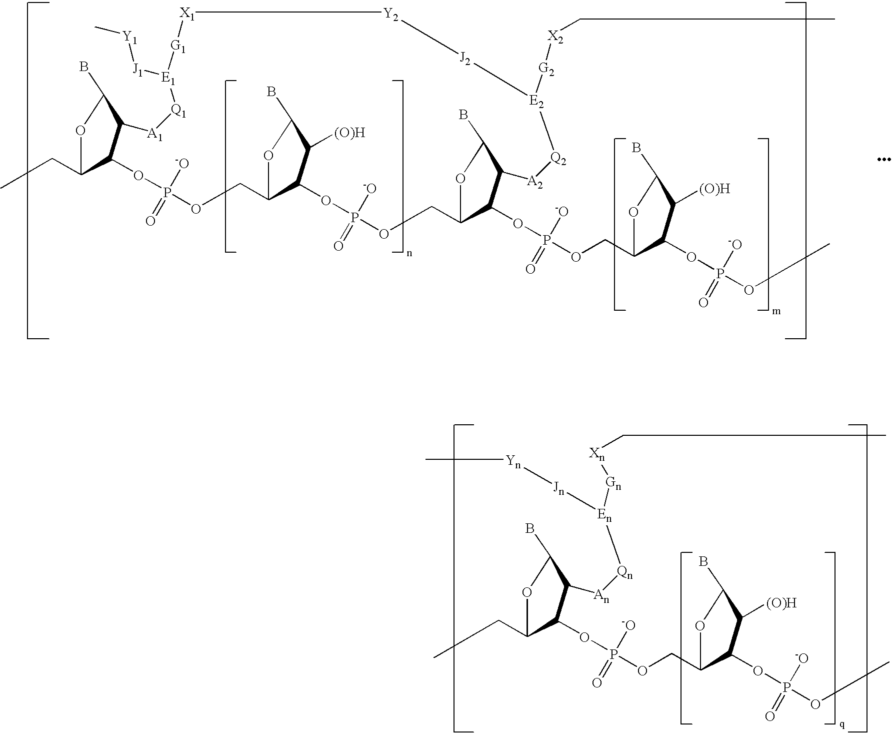

wherein:

A=a Group VI element selected from the group consisting of O, S, Se, and Te;

G, J, Q=a linker group selected from the group consisting of C₁-C₁₈ branched and straight chain alkyl groups, C₆-C₂₄ substituted and unsubstituted aromatic and heteroaromatic groups having from 1-3 hetero atoms (e.g., N, S, O) or halogen substitution, —O—, —S—, carbonyl, carboxyl, —Si(R)₂—, and —OSi(R)₂O—;

B=a nucleic acid base selected from the group consisting of U, T, A, G, C, and derivatives thereof recognizable to one skilled in the art as a nucleic acid “base”, and can be the same or different on different nucleotide units;

E=a symmetric or asymmetric atom center selected from the consisting of CR, N, NR+, phosphine, phosphine oxide, phosphate, phosphonate, phosphinate, phosphoramide, phosphonamide, and phosphinamide;

R=a terminal group selected from the groups consisting of H, C₁-C₁₈ branched and straight chain alkyl groups, C₆-C₂₄ substituted and unsubstituted aromatic, and heteroaromatic groups having from 1-3 hetero atoms (e.g., N, S, O) or halogen substitution;

The subscripts, e.g., 1, 2, n, etc., denote not only a sequence in the chain of units (brackets) forming a copolymer but also denote that the moieties designated by the letters, e.g., B, X, Y, etc., may or may not be the same from unit to unit.

The X-Y pair preferably forms amide, ester, phosphoester, or alkene bonds, such as from electrocyclic reactions. Most preferably, the X-Y pair forms an amide bond.

The polymer produced from desulfurization reaction of the polymer of formula (I) is presented below as formula (II)

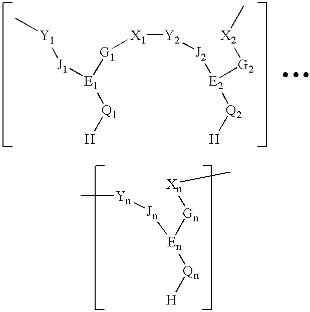

An example of the polymer of formula (I) is a DNA/polyamide polymer having the structure of formula (III) below.

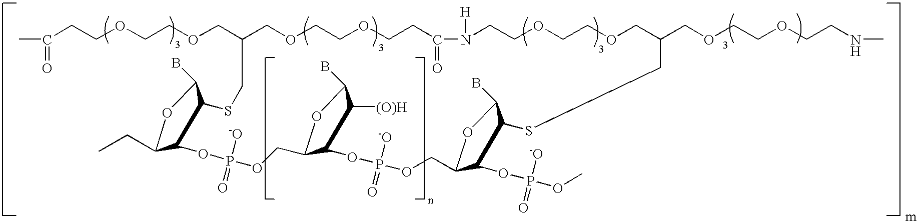

The polymer that would be produced from desulfurization reaction of formula (III) is shown below as formula (IV).

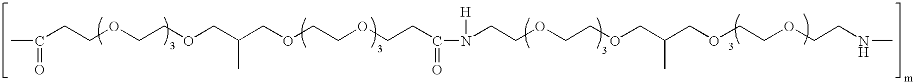

The present invention further provides a process for producing the polymer of formula (II) by using the nucleic acid translation system of the present invention to assemble a polymer of formula (I) and then forming/producing the polymer of formula (II) by desulfurization reaction.

Having now generally described the invention, the same will be more readily understood through reference to the following example which is provided by way of illustration and is not intended to be limiting of the present invention.

### EXAMPLE

### Experimental Methods

### Sequence Design

The sequences have been designed by applying the principles of sequence symmetry minimization, using the program SEQUIN (Seeman, 1982 and 1990).

### Synthesis and Purification

The strands were either synthesized on an Applied Biosystem 394 or an Expedite 8909, removed from the support, and deprotected using routine phosphoramidite procedures (Caruthers, 1985). Additional strands were purchased from IDT (Coralville, Iowa). Strands were purified using denaturing gel electrophoresis. Gels contained 10-20% acrylamide (19:1, acrylamide/bisacrylamide), 8.3 M urea and were run at 55° C. on a Hoefer SE 600 electrophoresis unit. Running buffer consisted of 89 mM Tris base, 89 mM boric acid, 2 mM EDTA at pH 8.0. The sample buffer contained 10 mM NaOH, 1 mM EDTA, 90% formamide and a trace amount of Xylene Cyanol FF tracking dye. Gels were stained with ethidium bromide, and the target band was excised and eluted in a solution containing 500 mM ammonium acetate, 10 mM magnesium acetate, and 1 mM EDTA. The eluates were subjected to extraction with n-butanol to remove ethidium bromide, followed by ethanol precipitation.

### Formation of Hydrogen-Bonded Complexes and Arrays

Complexes were formed by mixing a stoichiometric quantity of each strand as estimated by OD₂₆₀. Concentration of DNA and buffer conditions varied. Mixtures were annealed from 90° C. to room temperature during 40 h in a 2-liter water bath insulated in a styrofoam box. ‘Fast annealing’ consists of incubating the sample 5 min at 90° C., 15 min at 65° C., 20 min at 45° C., 20 min at 37° C. and 30 min at room temperature.

### Non-Denaturing Gel Electrophoresis

Annealed complexes, were run on non-denaturing gels to check for tile formation and stoichiometry. The systems were annealed at various DNA concentrations (0.1-3 uM) in 40 mM Tris-HCl, 20 mM acetic acid, 125 mM Mg Acetate, 2 mM EDTA. Tracking dye containing buffer, 50% glycerol, and a trace amount of Bromophenol Blue and Xylene Cyanol FF was added to the annealed sample before loading them on 6-8% acrylamide gels, containing their respective buffer. Gels were run on a Hofer SE-600 gel electrophoresis unit at room temperature, with the respective running buffer. After electrophoresis, the gels were stained with ethidium bromide.

### Ligation and Analysis.

The solution was brought to 1 mM in ATP and 10 units of T4 polynucleotide ligase (USB) were added. The ligation proceeded at 16° C. for 16 hours. Following ligation, the solution was heated at 90° C. for 5 minutes, and the ligation products were purified using 10% denaturing PAGE. The ligation products were sequenced to establish the correct assembly. A few missed or unknown bases are noted in the experimental sequencing, but these are far from the ligation points, and likely represent errors in the sequencing procedure.

### Radioactive Labeling.

Two pmol of an individual strand of DNA was dissolved in 10 μl of a solution containing 50 mM Tris HCl, pH 7.6, 20 μM spermidine, 10 mM MgCl₂, 15 mM dithiothreitol (DTT), and 0.2 mg/mL nuclease free bovine serum albumin (BSA) (US Biochemical) and mixed with 1 L of 1.25 mM γ-³²P-ATP (10 μCi/μL) and 3 units of T4 polynucleotide kinase (USB) for 2 h at 37° C. DNA was recovered by ethanol precipitation.

Results and Discussion

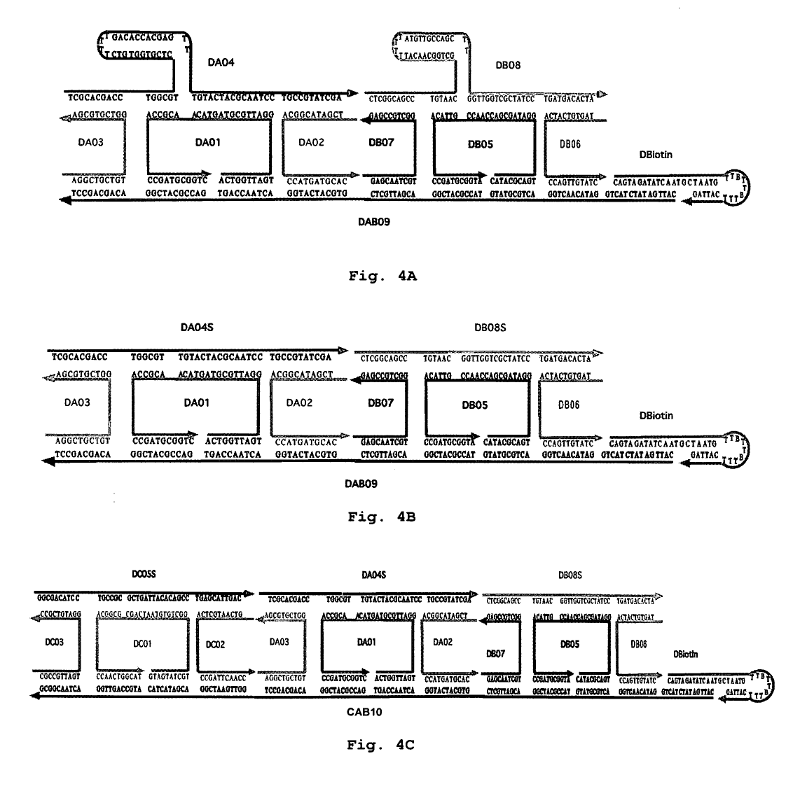

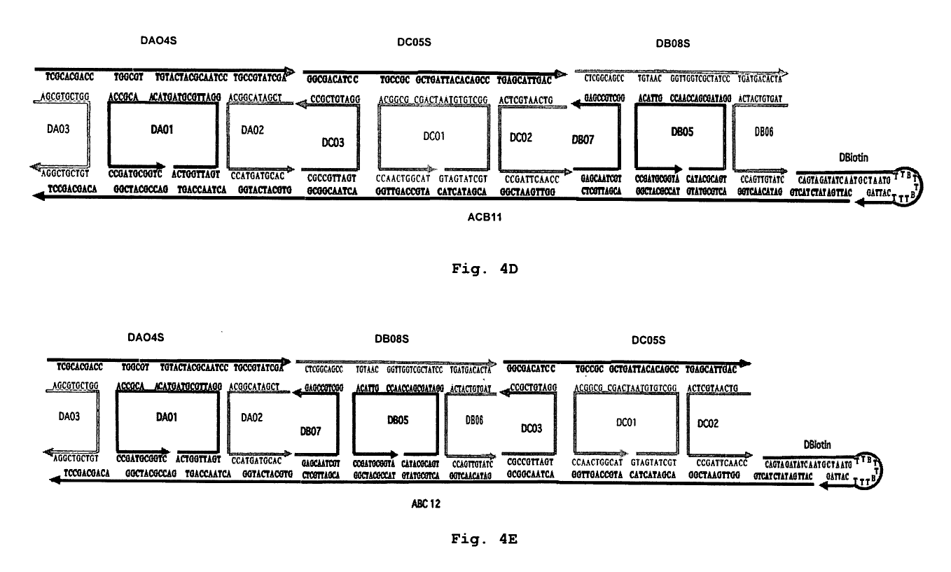

The experimental systems used in this Example are shown in FIGS. 4A-4E. FIG. 4A shows the first system that was used: Two DAE units connected laterally by the message strand on the bottom, labeled DAB09. To its right is a biotin-containing hairpin loop that terminates the assembly. It was initially believed that a biotin-based magnetic streptavidin bead purification would be needed in this system to eliminate incomplete assemblies, as was done in the previous system (Liao et al., 2004). However, that step proved not to be necessary; for convenience, the present inventors used the same biotinylated strand throughout this work, but never used a biotin-based purification. The hybridization procedure was refined based on the results obtained with this system: In the first attempts, the tiles were annealed separately, i.e., Tile A consisting of strands DA01 DA02, DA03 and DA04 and Tile B consisting of strands DB05, DB06, DB07 and DB08, following a fast annealing protocol, were then mixed together with strands Dbiotin and DAB09, heated to 40° C. and cooled to 16° C. followed by ligation of strands DA04 and DB08 (strand DB08 contains a phosphate group on its 5′ end). This protocol gave undesired products together with the expected one. So as to minimize undesired ligation products, all the strands were annealed together thereafter from 90° C. to 16° C. The sequencing of the AD product in FIG. 4A did not give good results; presumably this was due to the difficulty in completely denaturing the hairpins on both strands DA04 and D08, a necessary step during the PCR amplification done before sequencing. Even though the signal is very low starting from around the 40th base, the sequence is correct for the most part.

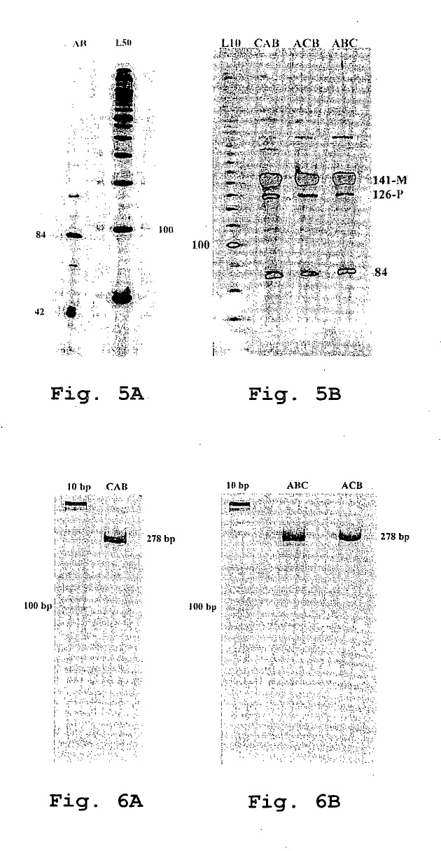

These problems led the present inventors to design a new system, illustrated in FIG. 4B; it is related closely to the first system, but it lacks the extra loops in the product strands. The complex forms well, as demonstrated by non-denaturing gel electrophoresis, and the target band containing 84 nucleotides is the primary product, although some higher bands are visible on the denaturing gel that characterizes the products of the ligation reaction (FIG. 5A). Most importantly, the sequence that was obtained is correct and is easy to read.

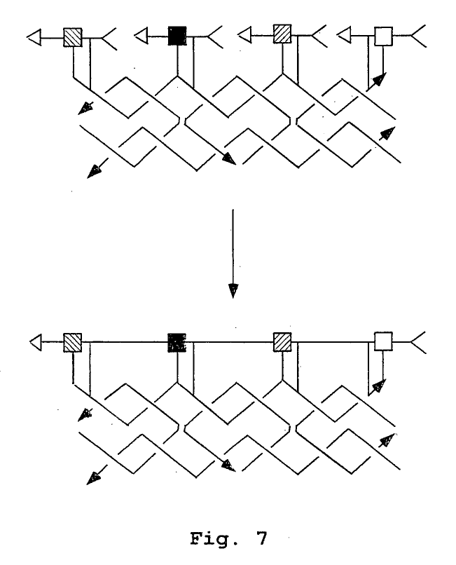

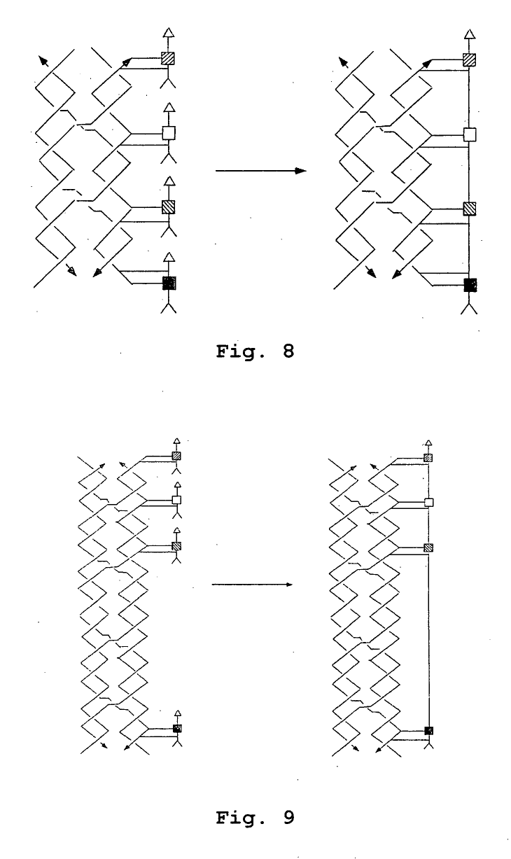

These preliminary results encouraged the present inventors to move onward to three-component systems, illustrated in FIGS. 4C, 4D and 4E. These three molecules represent permutations of the same three sequences in the message strand, which should lead to corresponding permutations of the product strands in the ligated material. The first question that is addressed here is whether the translation complexes form cleanly. FIGS. 6A and 6B contain non-denaturing gels illustrating that the hybridization products are concentrated into a single band of approximately the expected molecular weight. In general, this is taken to be an indication that the complex has formed well (Seeman, 2002). Smaller contaminants are just barely visible in each of the triple complexes, but these are not significant contributors to the overall population of molecules; this is why the biotin-based purification noted above was unnecessary. In each of the three cases shown in FIGS. 4C-4E, the target translation product was obtained as the major band on the denaturing gel analyzing the products of ligation. This gel is shown in FIG. 5B. As is characteristic of these systems (Seeman, 2002), there are some bands that represent failures of ligation. However, the key issue is whether the target product molecules have been assembled in the order prescribed by the message strand. In each case, the sequence of the product is the target dictated by the message. A simplified translation system combining the chemical simplicity of using conventional DNA with the simple translator method of Endo et al (2005) is described here. There is no ambiguity about the products at the level of a three-unit message. Although such translation systems are not likely to be involved in nucleic acid metabolism, it is worth pointing out that meiotic intermediates are DPE-type (Fu et al., 1993) DX molecules (Schwacha et al., 1995) that also have continuous strands analogous to the message and product strands discussed here. PX molecules, which have been suggested as being involved in the search for homology (Shen et al., 2004) in cellular systems have similar features.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Bang, D., Kent, S. B. H., A one-pot total synthesis of crambin, Angew. Chem. Int. Ed., 43:2534-8 (2004)
Caruthers, M. H., Science, 230:281-285 (1985)
Cello J, Paul A V, Wimmer E, Science, 297:1016-1018 (2003)
Endo, M; Uegaki, S.; Majima, T., Chem. Commun., 3153-3155 (2005)
Freier S, and Altmann K.-H., The ups and downs of nucleic acid duplex stability: structure-stability studies on chemically-modified DNA:RNA duplexes, Nucleic Acids Research, 25:4429-4443 (1997)
Fu et al., Biochemistry, 32:3211-3220 (1993)
Fu, T.-J.; Seeman, N. C. Biochem., 32:3211-3220 (1993)
Holliday, R., Genet. Res., 5:282-304 (1964)
Kanan, M. W., Rozeman, M. M., Sakurai, K., Snyder, T. M., Liu, D. R., Reaction discovery enabled by DNA-templated synthesis and in vitro selection, Nature, 431:545-549 (2004)
Liao, S; Seeman, N. C., Science, 306:2072-2074 (2004)
Mao, C.; Sun, W.; Shen, Z.; Seeman, N. C., Nature, 397:144-146 (1999)
Mukhopadhyay, P., Wu, A., Isaacs, L., Social self-sorting in aqueous solution, J. Org. Chem., 69:6157-6164 (2004)
Piccirilli et al., Nature, 343:33-37 (1990)
Rothemund P. W. K., Papadakis N, Winfree E, PLOS Biology, 2:2041-2053 (2004)
Rothemund, P. W. K.; Ekani-Nkodo, A.; Papadakis, N.; Kumar, A.; Fygenson, D. K.; Winfree, E. J. Am. Chem. Soc., 126:16344-16352 (2004)
Schwacha, A.; Kleckner, N., Cell, 83:783-791 (1995)
Seeman, N. C., J. Biomol. Str. & Dyns., 8:573-581 (1990)
Seeman, N. C., J. Theor. Biol., 99:237-247 (1982)
Seeman, N. C., Protocols in Nucleic Acid Chemistry, John Wiley & Sons, New York, Unit 12.1 (2002).
Shen, Z.; Yan, H; Zhang, X.; Seeman, N. C., J. Am. Chem. Soc., 126:1666-1674 (2004)
Winfree, E.; Liu, F.; Wenzler, L. A.; Seeman, N. C. Nature, 394:539-544 (1998)
Zhu, L., dos Santos, O, Seeman, N. C., Canary, J. W., Reaction of N3-Benzoyl-3′,5′-O-(di-tert-butylsilanediyl)uridine with hindered electrophiles: Intermolecular N3 to 2′O protecting group transfer, Nucleotides, nucleosides, and Nucl. Acids, 21:723-735 (2002)
Zhu, L., Lukeman, P. S., Canary, J. W. & Seeman, N. C., Nylon/DNA: Single-stranded DNA with covalently stitched nylon lining, J. Am. Chem. Soc., 125:10178-10179 (2003).
Zhu, L.; Lukeman, P. S.; Canary, J.; Seeman, N. C., J. Am. Chem. Soc., 125:10178-10179 (2003)

What is claimed is:

1. A nucleic acid-based translation system, comprising:
a nucleic acid template strand that serves as a message;
a plurality of nucleic acid product strands; and
a plurality of nucleic acid crossover strands capable of forming, with said nucleic acid template strand and said plurality of nucleic acid product strands, at least one nucleic acid multicrossover molecule,
wherein:
said nucleic acid crossover strands, that form at least one nucleic acid multicrossover molecule together with said nucleic acid template strand and said plurality of nucleic acid product strands, serve as a translation device to translate or decode the nucleotide sequence of said nucleic acid template strand serving as a message into said plurality of nucleic acid product strands;
said nucleic acid crossover strands that translate or decode said nucleic acid template strand into said plurality of nucleic acid product strands anneal with both said nucleic acid template strand and said plurality of nucleic acid product strands to form at least one nucleic acid multicrossover molecule;
each of said at least one nucleic acid multicrossover molecule comprises a first helix and a second helix that are parallel to each other;
each of said first and second helices contains a unidirectional nucleic acid strand disposed along its respective helical axis or alternatingly disposed along the helical axes of both of said first and second helices; and
one of said unidirectional nucleic acid strands of said first and second helices is said nucleic acid template strand and the other of said unidirectional nucleic acid strand of said first and second helices comprises said plurality of nucleic acid product strands translated or decoded from said nucleic acid template strand by said translation device of nucleic acid crossover strands, and wherein said plurality of nucleic acid product strands are capable of being ligated together into a chain of nucleic acid product strands.

2. The nucleic acid-based translation system of claim 1, wherein the nucleic acid is DNA.

3. The nucleic acid-based translation system of claim 1, wherein said at least one nucleic acid multicrossover molecule is a nucleic acid double crossover molecule.

4. The nucleic acid-based translation system of claim 3, wherein said nucleic acid double crossover molecule is a DAE molecule with said unidirectional nucleic acid strands of said first and second helices being antiparallel to each other.

5. The nucleic acid-based translation system of claim 3, wherein said nucleic acid double crossover molecule is a DPE molecule with said unidirectional nucleic acid strands in said first and second helices being parallel to each other.

6. The nucleic acid-based translation system of claim 3, wherein said nucleic acid double crossover molecule is a DPON molecule with said unidirectional nucleic acid strands in said first and second helices being parallel to each other.

7. The nucleic acid-based translation system of claim 3, wherein said nucleic acid double crossover molecule is a DPOW molecule with said unidirectional nucleic acid strands in said first and second helices being parallel to each other.

8. The nucleic acid-based translation system of claim 1, further comprising a nucleic acid strand that link together said nucleic acid template strand, said plurality of nucleic acid product strands and said plurality of nucleic acid crossover strands into at least one nucleic acid triple crossover molecule.

9. The nucleic acid-based translation system of claim 1, wherein said plurality of nucleic acid product strands have organic molecules appended on their backbones, said appended organic molecules serving as monomers and having reactive groups which can be reacted to polymerize the monomers together into a polymer chain.

10. The nucleic acid-based translation system of claim 9, wherein said appended organic molecules are a mixture of different organic molecules with compatible reactive groups.

11. The nucleic acid-based translation system of claim 9, wherein said polymer of appended organic molecules is a polymer of different monomeric units.

12. The nucleic acid-based translation system of claim 9, wherein said nucleic acid template strand serves as the nucleic acid template strand of a sequence of nucleic acid multicrossover molecules formed by the nucleic acid-based translation system.

13. A method for synthesizing a polymer sequence of organic molecules, comprising:
operating the nucleic acid-based translation system of claim 9 to produce a sequence of nucleic acid product strands with organic molecules appended on their backbones;
polymerizing the appended organic molecules together by their reactive groups to synthesize an appended polymer sequence of organic molecules.

14. The method of claim 13, further comprising cleaving the appended polymer sequence of organic molecules from the nucleic acid product strands.

15. The method of claim 13, further comprising ligating the nucleic acid product strands together into a continuous chain of nucleic acid product strands.

16. A method for decoding an encrypted message on a nucleic acid strand using the nucleic acid-based translation system of claim 1, comprising:
adding a set of nucleic acid crossover strands as decoder keys and a set of nucleic acid product strands as decoded unidirectional nucleic acid message strands to a nucleic acid template strand as an encrypted unidirectional nucleic acid message strand which contains the encrypted message in the form of the nucleotide sequence of the nucleic acid message strand, wherein the decoder keys to decode the encrypted message are either nucleic acid crossover strands that can anneal to both the encrypted nucleic acid message strand and the decoded nucleic acid message strands or a combination of nucleic acid crossover strands and a nucleic acid strand that link together the nucleic acid message, product and crossover strands into nucleic acid multicrossover molecules;
annealing the nucleic acid crossover strands, or a combination of nucleic acid crossover strands and a nucleic acid strand that link together the nucleic acid message, product and crossover strands into nucleic acid multicrossover molecules, as decoder keys, to the encrypted unidirectional nucleic acid message strand and the decoded unidirectional nucleic acid message strands to form at least one nucleic acid multicrossover molecule having at least two parallel helices, the encrypted unidirectional nucleic acid message strand being disposed in one or more helices along its length, and the decoded unidirectional nucleic acid message strands being disposed in the parallel helice(s) opposite from the encrypted unidirectional nucleic acid message strand; and
determining the decoded message from the decoded nucleic acid message strands.

17. The method of claim 16, wherein the nucleic acid is DNA.

18. The method of claim 16, further comprising ligating the decoded nucleic acid message strands into a continuous chain of decoded nucleic acid message strands before determining the decoded message from the continuous chain of decoded nucleic acid message strands.

19. The method of claim 18, further comprising denaturing the at least one nucleic acid multicrossover molecules to release the continuous chain of decoded nucleic acid message strands and isolating the released continuous chain of decoded nucleic acid message strands before determining the decoded message from the continuous chain of decoded nucleic acid message strands.

20. The method of claim 16, wherein said at least one nucleic acid multicrossover molecule is a nucleic acid double crossover molecule.

21. The method of claim 20, wherein said nucleic acid double crossover molecule is a DAE molecule with said unidirectional nucleic acid strands of said first and second helices being antiparallel to each other.

22. The method of claim 20, wherein said nucleic acid double crossover molecule is a DPE molecule with said unidirectional nucleic acid strands in said first and second helices being parallel to each other.

23. The method of claim 20, wherein said nucleic acid double crossover molecule is a DPON molecule with said unidirectional nucleic acid strands in said first and second helices being parallel to each other.

24. The method of claim 20, wherein said nucleic acid double crossover molecule is a DPOW molecule with said unidirectional nucleic acid strands in said first and second helices being parallel to each other.

25. The method of claim 16, wherein the at least one nucleic acid multicrossover molecule is a nucleic acid triple crossover molecule.
